(19) United States
(12) Patent Application Publication
(10) Pub. No.: US 2026/0102793 A1
(43) Pub. Date: Apr. 16, 2026

(54) METHODS OF WASHING A GRAIN PARTICLE MATERIAL AND/OR A STILLAGE COMPOSITION, AND RELATED SYSTEMS AND BIOREFINERIES

(71) Applicant: POET Research, Inc., Sioux Falls, SD (US)

(72) Inventors: Shannon S. Urban, Valley Springs, SD (US); Anna M. Andersen, Valley Springs, SD (US); Andrew J. Manning, Sioux Falls, SD (US); Steven T. Bly, Sioux Falls, SD (US); Stephen M. Lewis, Sioux Falls, SD (US); Jennifer Rebecca Headman, Sioux Falls, SD (US); Benjamin M. Gerdes, Yankton, SD (US); Kenny Dagvadorj Lee, Yankton, SD (US)

(21) Appl. No.: 19/359,336

(22) Filed: Oct. 15, 2025

Related U.S. Application Data
(60) Provisional application No. 63/707,857, filed on Oct. 16, 2024.

(51) Int. Cl. B08B 3/10; A23K 10/12; A62D 3/02; B08B 3/08; C12P 7/10

(52) U.S. Cl. CPC B08B 3/10; A23K 10/12; A62D 3/02; B08B 3/08; C12P 7/10; B08B 2220/00; B08B 2240/00

(57) ABSTRACT

Methods and systems for washing grain particle material and/or a stillage composition. Washing can be used to separate components such as one or more mycotoxins from the grain particle material and/or stillage composition. The methods include providing an aqueous composition that includes grain particle material or a stillage composition, and an aqueous liquid. The aqueous composition is separated into a solids fraction and at least one liquid fraction so that at least a portion of the components are separated from the solids fraction into the liquid fraction.

### CROSS-REFERENCE TO RELATED APPLICATION

This nonprovisional patent application claims the benefit of U.S. Provisional Patent Application Ser. No. 63/707,857, filed on Oct. 16, 2024, wherein said provisional patent application is incorporated herein by reference in its entirety.

### BACKGROUND

The present disclosure relates to washing grain particle material (e.g., grain flour) with an aqueous liquid such as water to reduce the concentration of one or more components (e.g., mycotoxins) in the grain particle material.

There is a continuing need for improved methods and system for reducing the concentration of one or more components that are present in whole grain.

### SUMMARY

The present disclosure includes embodiments of a method of washing a grain particle material. The method includes providing an aqueous composition that includes grain particle material and an aqueous liquid. The method also includes separating the aqueous composition into a solids fraction and at least one liquid fraction. The method also includes combining the solids fraction with water (e.g., tap water, backset, combinations of these, and the like) to form a slurry that includes grain starch.

The present disclosure also includes embodiments of a system configured to wash a grain particle material. The system includes a washing system, a separation system, and a mixing system. The washing system is configured to mix an aqueous composition that includes grain particle material and an aqueous liquid. The separation system is coupled to the washing system to receive the aqueous composition. The separation system is configured to separate the aqueous composition into a solids fraction and at least one liquid fraction. The mixing system is coupled to the separation system to receive the solids fraction and combine the solids fraction with water (e.g., tap water, backset, combinations of these, and the like) to form a slurry that includes grain starch.

The present disclosure also includes embodiments of a method of washing a grain particle material. The method includes providing an aqueous composition that includes grain particle material and an aqueous liquid. The method also includes separating the aqueous composition into a solids fraction and at least one liquid fraction. The method also includes treating the liquid fraction to reduce a concentration of one or more components in the liquid fraction.

The present disclosure also includes embodiments of a system configured to wash a grain particle material. The system includes a washing system, a separation system, and a wash-liquid treatment system. The washing system is configured to mix an aqueous composition that includes grain particle material and an aqueous liquid. The separation system is coupled to the washing system to receive the aqueous composition. The separation system is configured to separate the aqueous composition into a solids fraction and at least one liquid fraction. The wash-liquid treatment system is configured to treat the liquid fraction to reduce a concentration of one or more components in the liquid fraction. The wash-liquid treatment system is also configured to receive at least a portion of the liquid fraction from the separation system.

The present disclosure also includes embodiments of a method of washing a grain particle material. The method includes providing an aqueous composition that includes grain particle material and an aqueous liquid. A weight ratio of the aqueous liquid to the grain particle material is 5:1 or more. The method also includes separating the aqueous composition into a solids fraction and at least one liquid fraction.

The present disclosure also includes embodiments of a system configured to wash a grain particle material. The system includes a washing system and a separation system. The washing system is configured to mix an aqueous composition that includes grain particle material and an aqueous liquid. The washing system is also configured to combine the aqueous liquid and the grain particle material in a weight ratio of 5:1 or more. The separation system is coupled to the washing system to receive aqueous composition. The separation system is configured to separate the aqueous composition into a solids fraction and at least one liquid fraction.

The present disclosure also includes embodiments of a method of washing a stillage composition. The method includes providing an aqueous composition that includes stillage composition and an aqueous liquid. The method also includes separating the aqueous composition into a solids fraction and at least one liquid fraction.

The present disclosure also includes embodiments of a system configured to wash a stillage composition. The system includes a washing system and a separation system. The washing system is configured to mix an aqueous composition that includes stillage composition and an aqueous liquid. The separation system is coupled to the washing system to receive the aqueous composition. The separation system is configured to separate the aqueous composition into a solids fraction and at least one liquid fraction.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 shows a process-flow schematic an embodiment of a remediate system according to the present disclosure;

FIG. 2 shows a process-flow schematic of another embodiment of a remediate system according to the present disclosure;

FIG. 3 shows a process-flow schematic of a biorefinery coupled to a remediate system according to the present disclosure

FIGS. 4-8 show results from Example 2;

FIGS. 9 and 10 include results from Example 4;

FIGS. 11 and 12 include results from Example 6;

FIGS. 13 to 15 include results from Example 7;

FIG. 16 is a graph of % DON reduction versus UV exposure time from Example 8;

FIG. 17 is a graph of % DON reduction versus UV trial time from Example 9;

FIG. 18A is a graph of fermentation results from Example 10;

FIG. 18B is a bar chart of DON concentration in DDGS produced from Example 10; and

FIG. 19 is a graph of % DON reduction versus UV exposure time from Example 11.

### DETAILED DESCRIPTION

The present disclosure relates to systems configured to separate one or more components (e.g., one or more mycotoxins) from a grain particle material and/or one or more stillage compositions, and related methods. Such a system can also be referred to as a remediation system. “Remediation” refers to reducing the concentration of one or more undesirable components (e.g., microbes, organic acids, mycotoxins, and the like) that may be present in a grain particle material and/or in one or more stillage compositions. As discussed in more detail below, such a system can be used in a biorefinery.

As used herein, a biorefinery refers to a facility that can produce one or more bioproducts by converting plant-based feedstock via one or more physical processes, one or more chemical processes, one or more bioprocesses, and combinations thereof. Non-limiting examples of biorefineries include grain elevators, co-ops, grain handlers, dry mills, wet mills, biofuel production facilities, soy processing facilities, and the like. A bioproduct refers to a product derived from a biological, renewable resource. For example, a bioproduct can be a component of plant-based feedstock that is liberated from the plant-based feedstock (e.g., corn oil from corn grain) and/or can include a chemical (“biochemical” or “target biochemical”) that is produced by a biocatalyst (e.g., microorganism and/or enzyme) such as, for example, alcohol produced by yeast fermenting sugar. Non-limiting examples of bioproducts produced in a biorefinery include one or more of fuel, feed, food, pharmaceuticals, beverages and precursor chemicals. In some embodiments, a bioproduct includes, among others, one or more monomeric sugars, one or more enzymes, one or more plant oils, one or more alcohols (e.g., ethanol, butanol, and the like), one or more biogases (e.g., methane), fungal biomass, amino acids, and one or more organic acids (e.g., lactic acid), animal feed (e.g., distiller's wet grains (DWS), distiller's dried grains (DDG), distiller's dried grains with solubles (DDGS), grain distillers yeast (GDY), grain distillers dried yeast (GDDY)), and combinations thereof.

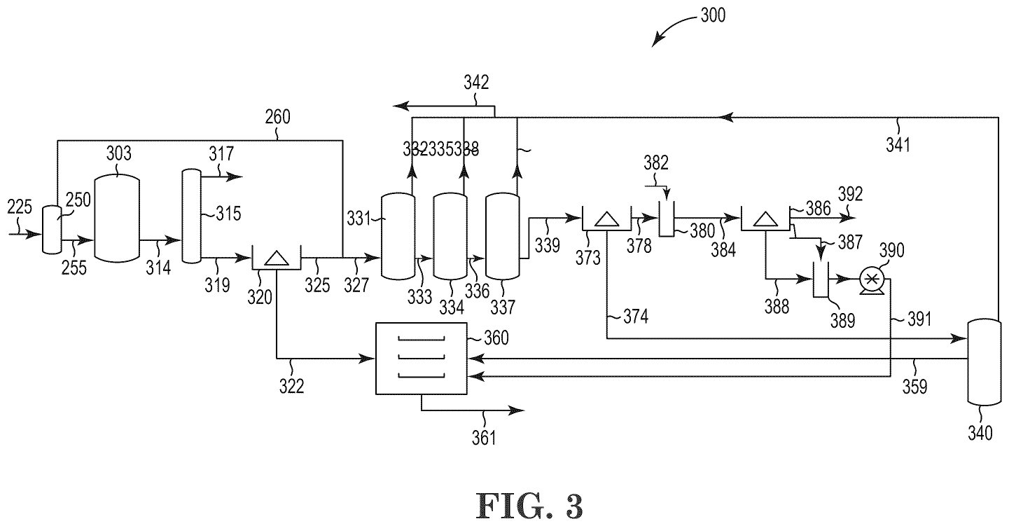

A biorefinery process composition is produced at a biorefinery. In some embodiments, a biorefinery process composition can include at least one composition to be used for fermentation (e.g., grain particle material, slurry) and/or produced by fermentation (e.g., beer), at least one stillage composition, and combinations thereof. As used herein, a “stillage composition” refers to a back-end composition of a fermentation process after separating (e.g., via distillation) one or more bioproducts from beer to form at least one target bioproduct stream (e.g., ethanol) and one or more co-product streams (e.g., whole stillage). A stillage composition can include whole stillage, at least one stillage composition derived from whole stillage, and combinations thereof. In some embodiments, whole stillage is derived from distilling beer in a dry-grind corn ethanol process/biorefinery. Non-limiting examples of a stillage composition derived from whole stillage include thin stillage, concentrated thin stillage (syrup), defatted syrup, defatted emulsion, clarified thin stillage, distiller's oil, DWS, DDG, DDGS, GDY, GDDY, and the like. Defatted syrup and defatted emulsion are examples of stillage compositions that remain after fat (e.g., corn oil) has been separated from syrup and emulsion, respectively, and can be referred to as “defatted stillage compositions.” Non-limiting illustrations showing one or more of the above-mentioned stillage compositions are shown in each of FIG. 3 (discussed below). A non-limiting example of making grain distillers dried yeast (GDDY) is described in U.S. Pub. No.: 2022/0015381 (Rindsig et al.), wherein the entirety of said patent publication is incorporated herein by reference.

A remediation system according to the present disclosure includes a washing system configured to wash at least a portion of one or more components from a biorefinery process composition. Such components include undesirable components that impact processing in a biorefinery and/or that impact the quality of a product output. Non-limiting examples of undesirable components include components that are present in incoming raw materials such as whole grain or make-up water and/or that are produced during processing. Non-limiting examples of undesirable materials that may be present in incoming raw materials include one or more mycotoxins, one or more microbes (e.g., lactic acid bacteria), and the like. Non-limiting examples of undesirable materials that may be produced during processing include one or more one or more organic acids (e.g., lactic acid) produced by microbes, and the like. Unless otherwise noted, for illustration purposes, the discussion below will refer to mycotoxins as an example of a component that can be washed away using washing systems and/or methods according to the present disclosure. However, it is understood that washing systems and/or methods according to the present disclosure can be used to wash away one or more other undesirable components instead of or in addition to one or more mycotoxins. In some embodiments, a remediation system according to the present disclosure includes a washing system configured to mix an aqueous composition that includes at least grain particle material; one or more mycotoxins; and an aqueous liquid. In some embodiments, a remediation system according to the present disclosure includes a washing system configured to mix an aqueous composition that includes at least a stillage composition; one or more mycotoxins; and an aqueous liquid. Unless otherwise noted, for illustration purposes, the discussion below will refer to washing away one or more components from a grain particle material using washing systems and/or methods according to the present disclosure. However, it is understood that washing systems and/or methods according to the present disclosure can be used to wash away one or components from a stillage composition instead of or in addition to a grain particle material. As used herein, an “aqueous composition” that includes aqueous liquid/water (e.g., tap water, backset, combinations of these, and the like) and grain particle material can also be referred to as a “slurry.”

Grain particle material is formed by exposing whole grain to one or more particle size reduction processes. At least a portion grain particle material can be used to form one or more biorefinery compositions. In some embodiments, the grain particle material can function as a carbon source and/or a nutrient source in fermentation, and can be used to form a fermentable composition. For example, a grain particle material can include one or more components that are utilized by a microorganism to produce one or more bioproducts via a bioprocess. Non-limiting examples of whole grains include corn, sorghum, wheat, rice, barley, oats, millet, combinations of these, and the like.

A particle-size reduction system can be used to process a feedstock including whole grain into grain particle material. A particle-size reduction system can include one or more size-reduction devices to reduce the size of whole grain and/or further reduce the size of ground grain that has previously been reduced in size. For example, a particle-size reduction system can include a coarse-grinding process and a fine-grinding process. A coarse-grinding process involves initially passing whole grain through roller mills and/or hammer mills to break the kernels into smaller pieces. This coarse grinding makes it easier for the subsequent grinding stages. Fine grinding involves passing the coarsely ground grain through a finer milling stage.

This can involve multiple passes through roller mills, hammer mills, or impact mills until the grain is reduced to the desired particle size.

Methods for reducing the size of feedstock such as whole grain and/or previously ground grain include dry milling such as passing the feedstock through one or more hammer mills, ball mills, roller mills, and/or other type of milling device to form grain flour, or simply flour. In some embodiments, a grain particle material can be formed to have a desired particle size distribution. For example, 50% by weight or more, 60% by weight or more, 70% by weight or more, 80% by weight or more, 90% by weight or more, or even 95% by weight or more of the grain particle material can have a particle size of 500 microns or less. If a stillage composition is derived from such grain particle material, and if the stillage composition is washed according to the present disclosure then the stillage composition would be expected to have a particle size that corresponds to the same or less than the particle size of the grain particle material. Sieves and screens can be used to control the particle size distribution of the grain particle material. Methods for reducing the size of feedstock also include wet milling such as passing a ground grain slurry through one or more mills such as a disc mills, roller mills, colloid mills, ball mills, and/or or other type of milling device.

It is noted that prior to particle-size reduction, whole grain can be cleaned to remove debris and/or surface impurities debris like stones, dust, soil, plant debris, which can interfere with the particle-size reduction process. Cleaning whole grain prior to particle-size reduction includes one or more of pre-cleaning, magnetic separation, screening, air aspiration, dehulling, combinations of these, and the like. Pre-cleaning refers to removing large debris like stalks, cobs, and stones, prior to the main cleaning. Magnetic separation refers to passing whole grain through magnetic separators to remove metallic impurities and foreign objects, which can damage particle-size reduction equipment. Screening refers to passing whole grains through vibrating and/or rotary screens to separate smaller impurities like dust, broken grains, and fine particles. Large screens can be used to separate relatively larger debris, while small screens can be used to separate fine particles. In some embodiments, relatively high levels of mycotoxin are associated with ground corn fines and separating the ground corn fines may help separate at least a portion of mycotoxins from the process of making ethanol in a dry-grind ethanol facility. Air aspiration or classifying refers to using an air stream in an aspirator or classifier, respectively, to remove lighter impurities like dust, husks, and chaff. Whole grains may also pass through a destoner, which uses differences in density to remove heavier impurities such as stones and glass. In some embodiments, whole grain may be dehulled to remove the outer husk. This is more common with whole grains other than corn, such as barley.

According to one aspect of the present disclosure, after forming a grain particle material, the grain particle material can then be washed in a washing system according to the present disclosure to separate one or more mycotoxins from the grain particle material and reduce the concentration of the one or mycotoxins in the grain particle material.

Mycotoxins are toxic fungal metabolites that are organic compounds of low molecular weight, often found in agricultural products that are characterized by their ability to cause health problems for humans and/or animals.

A mycotoxin can be found can be found both on the surface and inside of a whole grain. For example, a fungus that produces a mycotoxin can infect a whole grain on the surface, particularly in the outer layers of the whole grain. This initial infection can occur through damaged areas of the plant or during wet, humid conditions that favor fungal growth. Once the fungus infects the corn, it can penetrate deeper into the whole grain, allowing mycotoxin to accumulate not only on the surface but also inside the whole grain. The interior contamination occurs as the fungus grows within the grain tissue.

By washing a grain particle material according to the present disclosure, instead of or in addition to washing a whole grain, the concentration of mycotoxin can be sufficiently reduced, thereby permitting whole grain to be utilized that may otherwise be rejected because of high levels of one or more mycotoxins that cannot be sufficiently removed from or treated in the whole grain. Also, washing grain particle material according to the present disclosure to reduce mycotoxin levels to acceptable levels can do so without impacting fermentability of the grain particle material to an undue degree. For example, washing grain particle material according to the present disclosure can avoid washing away an undue amount of starch away from the grain particle material because the temperature of the aqueous liquid used as a washing liquid is low enough that it does not solubilize the starch to an undue degree. Also, if desired, washing a grain particle material according to the present disclosure can be performed alone or in combination with one or more other mycotoxin remediation techniques. For example, if washing a grain particle material according to the present disclosure reduces the level of one or more mycotoxins to a desired level, then one or more other mycotoxin treatments can be avoided that may introduce one or more undesirable chemical compounds. Alternatively, by washing a grain particle material according to the present disclosure to reduce the level of one or more mycotoxins to a certain level, then one or more other mycotoxin treatments that may otherwise be undesirable may be used in a manner that avoids the introduction of an undue concentration of one or more undesirable chemical compounds.

Non-limiting examples of mycotoxins include compounds such as aflatoxins, ochratoxins, patulin, fumonisins, zearalenones, trichothecenes, and combinations thereof. They are produced for example by different Fusarium, Aspergillus, Penicillium and Alternaria species.

Examples of trichothecene mycotoxins include T-2 toxin, HT-2 toxin, isotrichodermol, diacetoxyscirpenol (DAS), 3-deacetylcalonectrin, 3, 15-dideacetylcalonectrin, scirpentriol, neosolaniol; 15-acetyldeoxynivalenol, 3-acetyldeoxynivalenol, nivalenol, 4-acetylnivalenol (fusarenone-X), 4, 15-diacetylnivalenol, 4,7,15-acetylnivalenol, and deoxynivalenol (DON, also known as vomitoxin), and their various acetylated derivatives. The most common trichothecene in Fusarium head blight is deoxynivalenol produced for example by Fusarium graminearum and Fusarium culmorum.

One or more aqueous liquids can function as a “washing” liquid and be combined with to “wash” and separate one or more mycotoxins from grain particle material. A mycotoxin may be separated from the grain particle material by one or more mechanisms. For example, a mycotoxin may be separated, at least in part, by mechanical action during washing due to washing equipment; mechanical action during separating due to separating equipment; physical interaction between the aqueous liquid and the grain particle material; combinations of these, and the like. As another example, one or more mycotoxins may be water-soluble mycotoxins and may be separated, at least in part, by being solubilized into an aqueous liquid.

Non-limiting examples of an aqueous liquid include water (e.g., tap water), one or more process streams in a biorefinery, and combinations thereof. Non-limiting examples of process streams include treated wash liquid (discussed below), thin stillage, distillate, side-stripper bottoms, evaporator condensate, combinations of these, and the like. In some embodiments, the temperature of a process stream may be adjusted (e.g., cooled) prior to be used as an aqueous liquid for washing according to the present disclosure. For example, as discussed herein throughout, the temperature of the aqueous liquid can be relatively low enough so that it does not solubilize an undue amount of starch in the grain particle material during washing. An aqueous liquid according to the present disclosure is mostly water. For example an aqueous liquid is at least 50 percent by weight water, at least 90 percent by weight water, at least 95 percent by weight water, or even at least 99 percent by weight water.

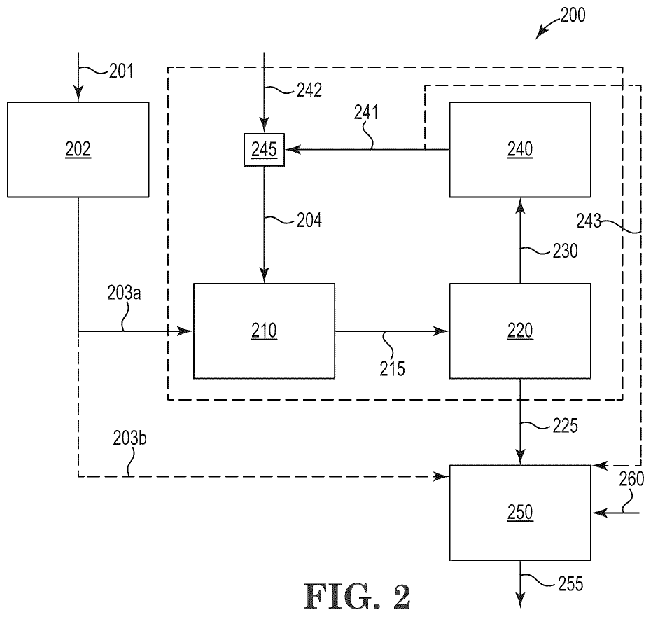

The temperature of the aqueous liquid may depend on whether a grain particle material is being washed or whether a stillage composition is being washed. For example, because a grain particle material has native components such as starch, which may be desired for fermentation, the temperature of the aqueous liquid when it is combined with grain particle material may be at a temperature that facilitates separation of one or more undesirable components, such as one or more mycotoxins, from the grain particle material while not separating desirable grain components, such as starch for fermentation, to an undue degree. In some embodiments, the temperature of the aqueous liquid when it is combined with grain particle material is below a starch-gelatinization temperature of the grain starch. For example, when washing grain particle material the aqueous liquid can be at a temperature of 55° C. or less, 50° C. or less, 40° C. or less, 35° C. or less, 30° C. or less, 25° C. or less, or even 20° C. or less. In some embodiments, the aqueous liquid is a temperature from 0° C. to 35° C., 0° C. to 30° C., 5° C. to 30° C., or 10° C. to 30° C. In some embodiments, having an aqueous liquid at temperature that is on the high end of 0° C. to 35° C. may be desirable if the treated wash liquid (discussed below) has been treated with ozone and is recycled to the washing system because the half-life of ozone increases with temperature, and the presence of ozone during washing grain particle material may be undesirable because of one or more reactions ozone may have with non-mycotoxin components of the grain particle material such as grain oil. At the same time, having the temperature at 35° C. or less can avoid undue solubilization of starch. In some embodiments, if the level of desirable components (e.g., starch, protein, and the like) that are solubilized along with undesirable components is relatively high (e.g., due to a relatively high-temperature wash liquid), then at least a portion of the treated wash liquid (see, e.g., liquid fraction 243 in FIG. 2 and discussed below) can be fed forward to making a slurry with the separated solids fraction.

If desired, the temperature of the aqueous liquid provided to and/or present in a washing system can be controlled via one or more heat exchangers and the like.

A washing system according to the present disclosure is configured to contain the aqueous liquid and the grain particle material in a weight ratio that facilitates washing the grain particle material to separate one or more mycotoxins therefrom. In some embodiments, a washing system according to the present disclosure is configured to contain an aqueous liquid and the grain particle material in the weight ratio of 2:1 or more, 3:1 or more, 4:1 or more, 5:1 or more, 6:1 or more, 7:1 or more, 8:1 or more, 9:1 or more, or even 10:1 or more, during washing.

The aqueous liquid and grain particle material can be combined together prior to and/or inside washing equipment. How and when the aqueous liquid and grain particle material are combined can depend on the washing method and/or washing equipment selected. Aqueous liquid can be used to wash grain particle material in a co-current manner or counter-current manner. Co-current washing is a process where the aqueous liquid (washing liquid) and the grain particle material to be washed move in the same direction through the washing system. This contrasts with counter-current washing, where the aqueous liquid (washing liquid) and the grain particle material move in opposite directions.

A washing system can include one or more units of washing equipment in series and/or parallel that can contain an aqueous liquid and the grain particle material in a weight ratio and be operated in a manner that facilitates separating one or more mycotoxins to a desired level while not separating one or more grain components (e.g., starch) to an undue degree.

A washing system can wash via “dilution” washing and/or “displacement” washing. In displacement washing, aqueous liquid (washing liquid) is introduced at one side of the solid material and displaces the dirty or contaminated liquid trapped within the solid matrix. An example of displacement washing is by dispensing the washing liquid onto the grain particle material while it is on a filter belt. Dilution washing involves mixing the solid material with a large volume of washing liquid to dilute the one or more components (e.g., mycotoxins) to be separated from the solid material such as grain particle material. The mixture is then separated into the “washed” solid fraction and the “dirty” liquid fraction. Dilution water tends to use relatively more washing liquid than displacement washing.

Non-limiting examples of washing equipment include slurry tanks, paddle mixers, ribbon blenders, high shear mixers, colloid mills, agitated reactors, continuous mixers, static mixers, turbine mixers, pug mills, and the like.

The dwell time of grain particle material in a unit or units of washing equipment in series can depend on variety of factors such as the target level of separation of the one or components, the weight ratio of aqueous liquid to grain particle material, the selected washing equipment, whether mixing of the aqueous composition is performed, combinations of these, and the like.

For example, in some embodiments, for water-soluble components such as one or more mycotoxins, a relatively short dwell time permits the grain particle material to become wet and solubilize the components to be separated. In some embodiments, the dwell time of grain particle material in a unit or units of washing equipment in series is at least 30 seconds, at least 1 minute, at least 2 minutes, at least 5 minutes, at least 10 minutes, at least 15 minutes, at least 20 minutes, at least 30 minutes, or even at least 60 minutes. In some embodiments, the dwell time of grain particle material in a unit or units of washing equipment in series is from 5 minutes to 120 minutes, from 5 minutes to 60 minutes, from 10 to 25 minutes, or even from 1 minute to 15 minutes. If mixing is included in the washing equipment, the aqueous composition can be mixed for a time period that corresponds to the dwell time.

After washing a grain particle material in a washing system, the aqueous composition that includes the grain particle material can be separated in a separation system into a solids fraction and at least one liquid fraction. The separation system is coupled to the washing system to receive the aqueous composition directly or indirectly. The solids fraction includes a majority of the suspended solids from the aqueous composition. Percent “total solids” content of an aqueous composition that is to be washed refers to the mass of solids that remain constantly after a sample of the aqueous composition has been dried in an oven at 103° C. for 24 hours divided by the mass of the sample of aqueous composition, and then multiplied by 100. The solids in the percent total solids content includes dissolved solids and suspended solids. Suspended solids refer to solids that remain intact in water and are not dissolved in water. Non-limiting examples of suspended solids include grain fiber particles, one or more grain fats (grain oils), one or more proteins, and the like. Protein can include grain protein. Dissolved solids refers to material in a sample that is dissolved in water and passes through a 0.2 micron filter, but that remains after the sample is dried. Non-limiting examples of dissolved solids include proteins, one or more vitamins, one or more minerals, one or more saccharides, and the like. A non-limiting example of a dissolved protein includes water-soluble corn gluten protein. The liquid fraction includes mostly water. The liquid fraction can also include some suspended and dissolved solids.

Notably, the liquid fraction includes one or more undesirable components such as mycotoxins that have been separated from the grain particle material or stillage composition to reduce the concentration of the one or more mycotoxins to a target level. The liquid fraction can also contain one or more grain components such starch, fiber, protein, and the like that would desirably remain in the solids fraction, instead of the liquid fraction, for a variety of reasons. For example, grain components can be used by microorganisms in fermentation. As another example, grain components that remain after fermentation can be separated as co-products such as oil, DDGS, and GDDY. As yet another example, grain components present in the liquid fraction can interfere with treatments such as ultraviolet radiation and/or ozone gas. The amount of grain components that end up in the liquid fraction can depend on the temperature of the aqueous liquid (wash liquid), the weight ratio of aqueous liquid to grain particle material or stillage composition, and/or the separation technique.

In some embodiments, by increasing the percent total solids in the solids fraction, the “washing efficiency” can be increased because that corresponds to more mycotoxins being separated into the liquid fraction. In some embodiments, the separation system is configured to separate the aqueous composition so that the solids fraction has total solids content of 40 percent or more, 50 percent or more, or even 60 percent or more, by total weight of the solids fraction.

The solids fraction corresponds to the grain particle material after washing (grain particle material that has been washed) that has a reduced concentration of one or more components (e.g., mycotoxins) and that can used in a biorefinery, e.g., for fermentation. As mentioned above, the amount of components such as mycotoxins that end up in the liquid fraction can depend largely on the washing efficiency driven by how much liquid is separated from the solids fraction in the context of dilution washing. It also depends on the starting concentration of the components being separated from the grain particle material and/or stillage composition, and the wash liquid to grain particle material ratio. In some embodiments, washing a grain particle material according to the present disclosure can result in a 70% reduction, 80% reduction, or even a 90% reduction in the washed grain particle material. In terms of parts-per-million (ppm), washing a grain particle material according to the present disclosure can result in grain particle material having a concentration of one or more mycotoxins of 1 ppm or less. However, it may not be necessary to reduce a mycotoxin concentration of, e.g., DON, to less than 1 ppm. For example, the FDA advises a DON limit for DDGS of 30 ppm, less than 10 ppm in a total ration for ruminating beef cattle and feed lot beef cattle older than four months. The FDA advises a DON limit for DDGS of 30 ppm, less than 5 ppm in a total ration for dairy cattle older than 4 months. The FDA advises a DON limit for grains and grain by-products of 5 ppm, less than 20% of diet for swine.

Remediation of mycotoxins according to the described process preserves the value of DDGS that would otherwise be degraded by the presence of mycotoxins in whole grain. Such remediation may also reduce input costs to a biorefinery operation by permitting the use of grain that has been discounted in price due to the presence of mycotoxins. The mycotoxin remediation system described herein can involve a relatively low capital investment and relatively low operating expense. The system may be monitored so that it uses water and operates equipment only when and only to the degree needed.

A separation system can include one or more units of separating equipment in series and/or parallel that can separate an aqueous composition that contain grain particle material and aqueous liquid into a solids fraction and at least one liquid fraction. Non-limiting examples of separating equipment include one or more solid-liquid separators chosen from one or more centrifuges (e.g., two-phase vertical disk-stack centrifuge, three-phase vertical disk-stack centrifuge, filtration centrifuge), one or more decanters (e.g., filtration decanters), one or more filters (e.g., tubular filter, fiber filter, rotary vacuum drum filter, filter device having one or more membrane filters), one or more screen devices (e.g., a “DSM” screen, which refers to a Dutch State Mines screen or sieve bend screen, and is a curved concave wedge bar type of stationary screen; one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof.

After separation, the solids fraction (grain particle material that has been washed and separated from the aqueous composition) can be packaged, stored, shipped, or transferred downstream in a biorefinery for further processing. An example of transferring a grain particle material downstream is illustrated with respect to FIG. 3, which is described below.

After separation, the liquid fraction can be treated to reduce the concentration of one or more mycotoxins in the liquid fraction. The liquid fraction can be treated in a wash-liquid treatment system that is configured to treat the liquid fraction, or portion thereof, to reduce a concentration of one or more mycotoxins in the liquid fraction. A variety of techniques can be used to treat the mycotoxins in the wash-liquid treatment system. Non-limiting examples of treating mycotoxins include chemical detoxification, enzymatic degradation, microbial degradation, high-voltage cold plasma, filtration, radiation, and combinations thereof.

Chemical detoxification includes chemical additives that can degrade mycotoxins, rendering them less harmful. Non-limiting examples of chemical additives include ammonia, oxidants, and combinations thereof. Ammonia treatment can break down certain mycotoxins.

Non-limiting examples of oxidants include hydroxyl radicals, hydrogen peroxide, ozone, superoxide anions, chlorine dioxide, persulfates, and combinations thereof. Hydroxyl radicals are potent oxidants in aqueous systems. They have a high reduction potential and can oxidize organic compounds by abstracting electrons, which can modify (breakdown) the molecular structure. While hydrogen peroxide is an oxidant on its own, it can be combined with catalysts (e.g., iron in the Fenton reaction) and/or UV radiation to produce highly reactive hydroxyl radicals, which are strong oxidants. Ozone can directly oxidize double bonds in organic molecules or generate secondary oxidants like hydroxyl radicals in the presence of water. For example, ozone can break down DON by targeting the epoxide ring and other reactive sites in its structure. The specific degradation products of DON after ozone treatment can vary depending on the conditions such as ozone concentration and exposure time. Because the degradation products from ozone treatment can vary, it can be advantageous to have a separate, dedicated wash-liquid treatment system for treating the liquid fraction instead of treating with ozone in the washing system. That way, the solids fraction can be separated from the aqueous composition before treating with ozone. The solids fraction can then be further processed downstream into one or more products such as one or more feed products. DON's toxicity is due at least to the double bond at carbon 9, the epoxy ring at carbon 12-13, and the hydroxyl group at carbon 3.

One of the primary targets of ozone in DON is the epoxide ring. Ozone can react with this group and break it down. The breakdown of the epoxy ring and other reactive sites in DON often results in the formation of simpler organic acids, ketones, and aldehydes. Ozonation products of DON in aqueous solution may be transitory depending on the ozone concentration and ozonation time. Most ozonation products are expected to be short lived under prolonged exposure to ozone. Most organic compounds will decompose into water and carbon dioxide if exposed to ozone long enough. As another example, ozone is effective against aflatoxins (e.g., aflatoxin B₁) by attacking the double bonds in the furan ring, leading to the formation of ozonides and subsequent degradation into less harmful compounds. Also, in addition to breaking down one or more mycotoxins while treating the liquid fraction, ozone can kill bacteria that may be present in the liquid fraction due to washing the grain particle material. Photocatalysis can use a photocatalyst like TiO2 in the formation of reactive oxygen species like hydroxyl radicals and superoxide anions, both of which are strong oxidants. Chlorine dioxide is another oxidant that is effective in breaking down organic compounds through electron abstraction and oxidation reactions. Persulfates can be activated by heat, UV light, or transition metals to produce sulfate radicals (SO₄•³¹ ) . These radicals are reactive and can degrade organic contaminants.

Microbial degradation includes certain bacteria, yeasts, and fungi that can degrade mycotoxins. For example, Trichoderma species can break down aflatoxins, and certain lactic acid bacteria can reduce the levels of zearalenone and ochratoxin.

Enzymatic degradation includes enzyme compositions that include at least one enzyme having an activity to detoxify at least one mycotoxin. Non-limiting examples of enzymes that can detoxify mycotoxins include epoxide hydrolases, carboxylesterases, laccases, deoxynivalenol epimerases, glutathione S-transferases (GSTs), aflatoxin-detoxifizyme, fumonisin esterases, and combinations thereof. Epoxide hydrolases can detoxify aflatoxins, specifically aflatoxin B1 (AFB1), by converting the harmful epoxide form into less toxic products. Carboxylesterases are involved in the detoxification of zearalenone (ZEN) by hydrolyzing the ester bonds and converting it to less toxic forms. Laccases are oxidoreductases that can break down ochratoxin A and other phenolic mycotoxins. Laccases oxidize the phenolic structures, reducing toxicity. Deoxynivalenol epimerases target deoxynivalenol (DON) and transform it into less toxic isomers.

In some embodiments, mycotoxin can be separated and concentrated relative to the liquid fraction from the separation system, followed by another treatment. For example, the liquid fraction from the separation system can be filtered (e.g., in a reverse osmosis filter) to separate and concentrate the mycotoxins in a retentate, followed by treating the retentate as described herein to detoxify the mycotoxins. This technique can be especially useful if the liquid fraction is going to be recycled within a biorefinery because the mycotoxin can be separated before detoxification, thereby avoiding having any degradation products being recycled along with the liquid fraction. Alternatively, the degradation products could be filtered from the liquid fraction after treatment, but prior to being recycled.

Detoxification via radiation includes exposing mycotoxin to radiation to degrade the mycotoxins, rendering them less harmful. A non-limiting example of radiation includes ultraviolet (UV) radiation. Ultraviolet treatment operates by exposing a stream such as the liquid fraction to high-energy UV radiation, typically in the UV-C range (around 254 nm). When mycotoxins are irradiated, they may undergo direct photolysis and/or indirect oxidation. Direct photolysis occurs where mycotoxin molecules absorb the UV photons, resulting in the breaking of chemical bonds and subsequent degradation into less harmful compounds. Instead of or in addition to direct photolysis, UV can be coupled with oxidizing agents such as hydrogen peroxide and/or ozone. In these processes, the UV light decomposes the oxidant to generate hydroxyl radicals, which are highly reactive that can rapidly oxidize mycotoxin molecules.

In some embodiments, a UV treatment system may include one or more UV lamps positioned within or adjacent to a flow-through reactor chamber through which a liquid fraction (e.g., used wash liquid) is recirculated. In some embodiments, a UV treatment system may include one or more UV lamps positioned within or adjacent to a reactor chamber filled with a liquid fraction (e.g., used wash liquid) that is static or mixed. The intensity of the lamps, influences the degradation efficiency. In some embodiment, each lamp can range from 10 to 1000 watts, or even from 100 to 1000 watts, depending on the flow rate and reactor size.

Treatment effectiveness is further affected by operating parameters such as temperature of the liquid to be treated (e.g., the “liquid fraction”), flow rate, and UV exposure time. In some embodiments, the liquid to be treated can be from 10° C. to 40° C. to facilitate lamp performance and reaction kinetics.

In some embodiments, a UV treatment system may employ multiple passes and/or recirculation of fluid to achieve sufficient cumulative UV dosage, with exposure times varying depending on mycotoxin load and target removal efficiency. Balance of these parameters can permit sufficient mycotoxin breakdown under practical operating conditions. As used herein, “UV exposure time” refers to the duration during which a given volume of liquid is subjected to ultraviolet radiation within an irradiation zone of a UV treatment system. UV exposure time does not include periods during which the liquid resides outside of the irradiation zone, such as in piping, tanks, or other non-irradiated portions of the system. Where the liquid undergoes multiple passes through the irradiation zone, the UV exposure time may be expressed as the cumulative duration of direct irradiation experienced across such passes. In some embodiments, UV exposure time can be from 1 second to 24 hours, from 30 seconds to 24 hours, or even from 1 minute to 120 minutes.

In addition to the degradation of mycotoxins, UV treatment systems may provide the benefit of microbial disinfection, e.g., due to undesirable microbes that may be present on incoming grain such as corn. The high-energy photons can penetrate microbial cell walls and damage nucleic acids, rendering bacteria, viruses, and protozoa unable to replicate. This dual action of chemical oxidation and pathogen inactivation can make UV-based systems particularly advantageous in treatment of, e.g., an aqueous liquid that is used for washing solids in a biorefinery as they can simultaneously reduce mycotoxin and provide effective disinfection. Advantageously, residual chemical disinfectants can be avoided if desired.

In some embodiments, after treating to reduce the concentration of one or more mycotoxins in the liquid fraction, the liquid fraction can be recycled for use as wash liquid in the washing system, as described above.

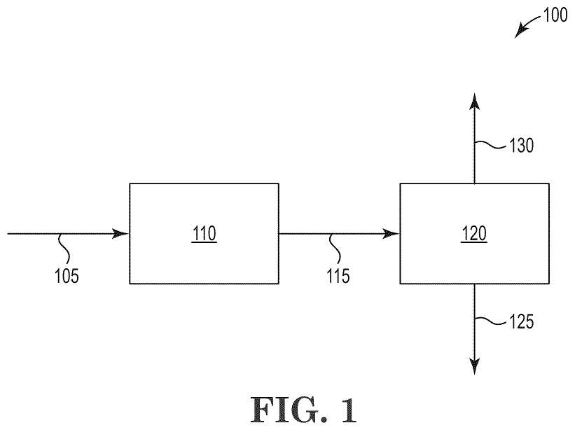

Non-limiting examples of a remediation system configured to separate one or more mycotoxins from a grain particle material are described below with respect to each of FIGS. 1 and 2.

Referring to FIG. 1, remediation system 100 includes a washing system 110 and a separation system 120, examples of which are described herein above. Washing system 110 is configured to mix an aqueous composition that includes grain particle material 103 having one or more mycotoxins and an aqueous liquid 104. The separation system 120 is coupled to the washing system 110 to receive the aqueous composition 115 after washing. The separation system 120 is configured to separate the aqueous composition into at least a solids fraction 125 and a liquid fraction 130.

FIG. 2 shows another example of a remediation system and how it could be coupled to an existing system or facility that utilizes the grain particle material after washing according to the present disclosure.

Referring to FIG. 2, remediation system 200 is indicated by the box in dashed lines and includes a washing system 210, a separation system 220, and a wash-liquid treatment system 240, examples of which are described herein above. Washing system 210 is configured to mix an aqueous composition that includes grain particle material 203a having one or more mycotoxins and an aqueous liquid 204. Grain particle material 203a is prepared from whole grain 201 that is processed in a particle-size reduction system 202, examples of which are described herein above. The separation system 220 is coupled to the washing system 210 to receive the aqueous composition 215 after washing. The separation system 220 is configured to separate the aqueous composition into at least a solids fraction 225 and a liquid fraction 230. The solids fraction 225, which corresponds to grain particle material that has been washed, has a reduced concentration of one or more mycotoxins and can be transferred to a process that utilizes the grain particle material. As shown in FIG. 2, solids fraction 225 is transferred to a slurry tank system 250 for use in a fermentation system, an example of which is described in more detail below with respect to FIG. 3. As shown in FIG. 2, solids fraction 225 is combined with backset 260 (thin stillage) to form a slurry 255. Optionally, or alternatively, solids fraction 225 can be combined with one or more other sources of water such as tap water and/or one or more process streams in a biorefinery, as described above with respect to “aqueous liquid,” including treated wash liquid such as liquid fraction 241, discussed below.

Liquid fraction 230 includes mycotoxins that have been separated from the grain particle material 203a. Remediation system 200 includes wash-liquid treatment system 240 that is configured to receive at least a portion of the liquid fraction 230 from the separation system 220, and reduce a concentration of the one or more mycotoxins in the liquid fraction 230. Examples of treating a liquid fraction to reduce a concentration of one or more mycotoxins in the liquid fraction are described herein above. As shown in FIG. 2, the liquid fraction 241 is wash liquid that has been treated after washing in the washing system to have a reduced concentration of one or more mycotoxins. As shown in FIG. 2, the liquid fraction 241 is recycled to washing system 210 to be used in aqueous liquid 204 as a wash liquid. As also shown in FIG. 2, liquid fraction 241 can be combined with one or more additional sources 242 of aqueous liquid such as tap water and/or one or more process streams in a biorefinery, as described above with respect to “aqueous liquid.” As shown, the liquid fraction 241 can be combined with one or more additional sources 242 of aqueous liquid in a buffer tank 245. Alternatively, the liquid fraction 241 can be combined with one or more additional sources 242 of aqueous liquid using simply piping and valves. Optionally, a portion 243 of liquid fraction 241 can be fed forward to slurry tank system 250 to form slurry 255, especially if there is a relatively high concentration of starch that is washed away from the grain particle material. Also, optionally, one or more additional sources 242 can also be treated with ozone to reduce any bacterial contamination before being introduced into washing system 210, which can help reduce organic acid that would otherwise be produced by the bacteria.

The remediation system 200 can be a portable system that can be moved around within a biorefinery and/or transported among multiple biorefineries to where it is to be used. For example, the remediation system 200 of FIG. 2 may be a skid mounted self-contained unit that is inserted into an existing process stream in an existing operation to provide for mycotoxin remediation. Alternatively, the remediation system 200 can be permanently installed.

FIG. 2 depicts remediation system 200 connected in line with a particle-size reduction system 202 and slurry tank system 250 of a biorefinery. Optionally, it is noted that a biorefinery may not prepare grain particle material, such as grain flour, onsite. Instead, the biorefinery may have grain particle material, such as grain flour, delivered to the biorefinery for processing.

The washing system 210 has an inlet that couples to an outlet of particle-size reduction system 202, and the separation system 220 has an outlet that couples to an inlet of the slurry tank system 250. As indicated by the dashed line, grain particle material 203b from particle-size reduction system 202 can bypass remediation system 200 using one or more valves to shut off grain particle material 203a and couple directly to slurry tank system 250 when mycotoxin remediation is determined to not be desired.

Optionally, whether or not to utilize a remediation system according to the present disclosure can be decided by determining if one or more mycotoxins are present in incoming whole grain and/or in one or more biorefinery process compositions.

Mycotoxin levels may be monitored and a controller may be used to control the remediation system using to reduce mycotoxin levels to an acceptable threshold (e.g. values as reported by the US Food & Drug Administration (FDA)). For example, one or more valves can be actuated to connect to a remediation system as well as the valves that control flowrate of aqueous liquid for wash liquid. One or more valves can also be actuated to control the introduction of an oxidant such as ozone into a wash-liquid treatment system.

Mycotoxin levels may be monitored at any point in the biorefinery operation. For example, the mycotoxin level in incoming whole grain may be monitored. In another example, the mycotoxin level may be monitored in DDGS and the mycotoxin remediation system controlled to maintain the DDGS mycotoxin level within an acceptable range. In another example the mycotoxin level may be monitored in a process stream upstream of the remediation system and/or in a process stream downstream of the remediation system. Alternatively, monitoring may be omitted and a remediation system may be operated to treat maximum expected values of mycotoxins. At times when no mycotoxin is present the remediation system can be idled to reduce costs. For example, whole grain may be tested prior to entry into the biorefinery, and in crop years and regions where mycotoxin are present above a threshold value the remediation system can be employed.

To monitor the mycotoxin levels in a biorefinery, a sample of incoming grain and/or one or more biorefinery process compositions can be manually obtained and tested. Testing of manual samples can be performed in a lab onsite and/or offsite from the biorefinery. Analytical techniques such as ELISA (Enzyme-Linked Immunosorbent Assay), HPLC (High-Performance Liquid Chromatography), and GC-MS (Gas Chromatography-Mass Spectrometry) are used to detect and quantify mycotoxin levels in grain and grain products. For example, VERATOX™ for DON 2/3 kit is a commercially available kit from NEOGEN™ for detecting Don in grains and grain products.

A remediation system may also or instead include one or more sensing or sampling systems (not shown) to automate the monitoring process. For example, the mycotoxin level in a biorefinery process composition at any point in a biorefinery operation may be monitored and communicated to the remediation system where it may be used to adjust one or more valves. The sensing or sampling systems can be avoided if desired. As mentioned above, grain and/or one or more biorefinery process compositions can be manually sampled and tested for the presence of mycotoxins.

FIG. 3 illustrates a non-limiting example of a biorefinery that is coupled to a remediation system according to the present disclosure. FIG. 3 illustrates an example of dry-grind corn ethanol biorefinery 300 (“biorefinery 300”) that produces stillage compositions described above. The biorefinery 300 includes a “front end” and a “back end.” The front end includes distillation system 315 and upstream from distillation system 315.

As shown in FIG. 3, the front end starts with adding solids fraction 225, which corresponds to “washed” grain particle material, from remediation system 200 to slurry tank system 250 to form slurry 255. Solids fraction 225 has a reduced concentration of one or more mycotoxins as compared to grain particle material 203a. One or more additional materials can be added to slurry tank system 250 to form slurry 255 (also referred to as a fermentable composition). Non-limiting examples of such materials include one or more microorganisms, one or more of enzymes, pH adjusters, antimicrobials (e.g., used against bacterial contaminants in yeast fermentation), and the like. Sulfuric acid is added to slurry tank system 250 to adjust the pH. One or more antimicrobials can be added to slurry tank system 250 to act against bacterial contaminants in yeast fermentation. One or more exogenous enzymes are added discretely to slurry tank system 250 to help break starch into monosaccharide such as glucose. One or more enzymes may be added for hydrolysis. In some embodiments, one or more enzymes used for hydrolysis can be produced by one or more microorganisms present in the slurry.

Slurry 255 is transferred to fermentation system 303. Fermentation system 303 converts slurry 255 into a post-fermentation broth 314 that includes at least one or more biochemical bioproducts. A bioproduct refers to a product derived from a biological, renewable resource.

For example, a bioproduct can be a component of biomass feedstock that is liberated from the biomass feedstock (e.g., corn oil from corn grain) and/or can include a chemical (“biochemical”) that is produced by a biocatalyst (e.g., microorganism and/or enzyme) such as, for example, alcohol produced by yeast fermenting sugar. Fermentation by a microorganism can produce biomass (e.g., single cell protein (SCP)), extracellular metabolites (e.g., alcohol such as ethanol), intracellular metabolites (e.g., enzymes), and combinations thereof. Non-limiting examples of such microorganisms include ethanologens, butanologens, combinations of these, and the like.

Exemplary microorganisms include yeast, algae, bacteria, and combinations thereof. For example, yeast may be used to convert the sugars to an alcohol such as ethanol. Suitable yeast includes any variety of commercially available yeast, such as commercial strains of Saccharomyces cerevisiae.

Fermentation system 303 can include one or more vessels that are adapted to expose a fermentable composition to conditions suitable for converting monosaccharide such as glucose to one or more bioproducts. As used herein, a “vessel” refers to any vessel that permits a bioproduct to be formed from a microorganism via fermentation. In some embodiments, a vessel can refer to a bioreactor adapted or configured to expose a fermentable composition to fermentation conditions. Non-limiting examples of vessels that can expose a fermentable composition to fermentation conditions include fermenters, beer wells, and the like. Two or more vessels may be arranged in any desired configuration such as parallel or series. As shown in FIG. 3, fermentation system 303 includes a single fermenter.

A fermentation system is configured to expose fermentable composition to fermentation conditions so that one or more microorganisms can convert one or more components in the fermentable composition such as sugars into one or more target bioproducts. Fermentation conditions include one or more conditions such as pH, time, temperature, aeration, stirring, and the like.

The pH of a fermentable composition can be at a pH that helps a microorganism produce one or more target bioproducts in a desired quantity. In some embodiments, the pH is greater than 3.5, e.g., from 3.5 to 7, from 3.5 to 5.5, or even from 3.5 to 4.5. Techniques for adjusting and maintaining pH include, e.g., adding one or more acidic materials and/or adding one or more basic materials. As mentioned above, sulfuric acid can be added to slurry tank system 250 to adjust the pH.

With respect to temperature and time, the contents of a fermentable composition can be maintained at temperature for time period helps a microorganism produce one or more target bioproducts in a desired quantity. In some embodiments, the temperature of a fermentable composition can be at a temperature in a range from 20° C. to 45° F., from 25° C. to 40° C., or even from 30° C. to 40° C. In some embodiments, fermentation can occur for a time period up to 72 hours, or even up to 96 hours. For example, fermentation can occur for a time period from 1 hour to 48 hours, from 2 hours to 48 hours, or even from 10 hours to 30 hours.

Fermentation can be performed under anaerobic conditions and/or aerobic conditions. For example, fermentation can be performed under aerobic conditions for at least a portion of the fermentation and performed under anaerobic conditions for another portion of fermentation.

Alternatively, all of fermentation can be performed under anaerobic conditions or under aerobic conditions. Anaerobic or aerobic conditions are selected based on the “target” biochemical or biochemicals chosen to be produced by a microorganism even though there may de minimis amounts of “non-target” biochemicals that are also produced by the microorganism. Anaerobic conditions means that the fermentation process is conducted without any intentional introduction of oxygen-containing gases such as with equipment like blowers, compressors, etc., that could operate to create an aerobic environment suitable for aerobic fermentation. It is noted that while simply stirring a fermentable composition to keep reactor contents homogenous may or may not introduce a de minimis amount of an oxygen-containing gas such as air in some embodiments, stirring alone may not create conditions that would be considered “aerobic conditions” as used herein. However, if desired, the contents of a fermenter could be mixed using appropriate equipment such that sufficient oxygen is introduced throughout the fermentable composition to create an aerobic environment suitable for aerobic fermentation (see below).

Aerobic conditions means that fermentation is performed with intentional introduction of one or more oxygen-containing gasses (“aeration”) to create an aerobic environment suitable for aerobic fermentation so that oxygen can be consumed by one or more microorganisms and selectively favor the production of enzymes via an aerobic metabolic pathway as compared to an anaerobic pathway which favors production of biochemicals (e.g., alcohol, organic acids, and the like). A fermentation system may incorporate aeration by including one or more blowers, spargers, gas compressors, mixing devices, and the like, that are in fluid communication with one or more fermentation vessels and that can introduce an oxygen-containing gas (e.g., air) into a fermentable composition during at least a portion of fermentation. For example, an oxygen-containing gas can be sparged into a fermentable composition so that the gas bubbles up and through the fermentable composition and oxygen transfers into the fermentable composition. As another example, an oxygen-containing gas can be introduced into the headspace of a fermenter so that the gas diffuses into the fermentable composition.

In some embodiments, an aerobic fermentation can be quantified by referring to a volumetric oxygen transfer coefficient (“kLa” constant) (hours(h)−1). The kLa constant describes how efficient oxygen is transferred from gas bubbles into the fermentable composition. The kLa constant depends on factors such as process conditions and geometry of a vessel used for fermentation (e.g., a fermenter). Process conditions include the volume flow of oxygen in the form of gas into a fermentable composition, pressure of the contents of a vessel, temperature of the contents of a vessel, and/or degree of mixing of the contents of a vessel. Geometry of a vessel used for fermentation includes height of the vessel. The kLa constant consists of the two coefficients. The mass transfer coefficient “kL,” which describes the transport of oxygen and gas into the liquid phase. And “a,” which refers to the gas-liquid exchange area per unit of liquid volume. Since it can be difficult to measure the kL and a value separately, they are combined into one parameter, the kLa constant. There are chemical, biological and physical methods that measure the kLa constant in a vessel used for fermentation. One method is referred to as the “static gassing-out” method, which involves installing an oxygen sensor in a vessel used for fermentation to measure the dissolved oxygen concentration in a liquid medium. The characterization is often done with water, but any liquid medium can be used. The oxygen concentration of the liquid medium is set to zero by degassing with nitrogen. Then, oxygen-containing gas is introduced or “gassed” (e.g., sparged) into the contents of the vessel again under process conditions using a defined gassing rate and stirrer speed. The oxygen sensor then measures the saturation process and the kLa can be determined. In some embodiments, a fermentation vessel operating under aerobic conditions has a kLa constant greater than 0.2, greater than 0.25 or even greater than 0.3 (e.g., from 0.3 to 5, or even from 0.35 to 3).

Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, viscosity, and the like. As mentioned above, in addition to mixing the contents of a composition, mixing can be selected, if desired, to intentionally incorporate oxygen to a fermentable composition to facilitate aerobic fermentation.

A fermentation system can be operated according to batch fermentation, fed-batch fermentation, or continuous fermentation (continuous feed and discharge from a vessel such as a fermenter).

Also, a fermentation system can conduct fermentation sequentially or simultaneously with respect to a polysaccharide hydrolysis/saccharification process (e.g., jet-cooking and/or enzymatic hydrolysis). Saccharification and fermentation can occur simultaneously according to what is known as “simultaneous saccharification and fermentation” (“SSF”). Sequential hydrolysis and fermentation can also be referred to as separate hydrolysis and fermentation (SHF).

An example of an SSF is described below in the context of a starch-containing grain such as corn. A slurry (“grain mash composition”) can be combined with a microorganism to form a fermentable composition so that at least a portion of starch in the fermentable composition is hydrolyzed by one or more enzymes to produce monosaccharides. As the monosaccharides are produced, they can be metabolized by a microorganism into a target biochemical product. For example, sugar (glucose, xylose, mannose, arabinose, etc.) that is generated from saccharification can be fermented into one or more biochemicals (e.g., butanol, ethanol, and the like).

Alternatively, an SHF process may include a dedicated saccharification process that is separate from a fermentation process (either in the same or separate vessel). For example, after forming an aqueous slurry that includes the biomass feedstock (e.g. corn material from a milling system) the aqueous slurry can be subjected to saccharification in one or more slurry tanks to break down (hydrolyze) at least a portion of the polysaccharides, e.g. starch, cellulose, hemicellulose, etc., into oligosaccharides and/or monosaccharides (e.g. glucose, xylose, mannose, arabinose, etc.) that can be used by microorganisms (e.g., yeast) in a subsequent fermentation process.

Saccharification can be performed by a variety of mechanisms. For example, heat and/or one or more enzymes can be used to form one or more monosaccharides by saccharifying one or more oligosaccharides and/or one or more polysaccharides that are present in a polysaccharide such as starch. In some embodiments, a relatively low temperature saccharification process (whether used in SSF or SHF) involves enzymatically hydrolyzing at least a portion of starch in an aqueous slurry at a temperature below starch gelatinization temperatures, so that saccharification occurs directly from the raw native insoluble starch to soluble glucose while bypassing conventional starch gelatinization conditions, which are typically in a range of 57° C. to 93° C. depending on the starch source and polysaccharide type. In some embodiments, saccharification includes using one or more enzymes (e.g., alpha-amylases and/or gluco-amylases) to enzymatically hydrolyze at least a portion of the starch in the aqueous slurry at a temperature of 40° C. or less to produce a slurry that includes glucose. In some embodiments, enzymatic hydrolysis occurs at a temperature in the range of from 25° C. to 35° C. to produce a slurry that includes glucose.

After fermentation, one or more bioproducts can be separated from post-fermentation broth 314 to form at least one target bioproduct stream (e.g., ethanol) and one or more co-product streams (e.g., whole stillage). One or more bioproducts produced in the fermentation system 303 can be separated from the post-fermentation broth 314. Referring to FIG. 3, a distillation system 315 is in fluid communication with the fermentation system 303 to receive a post-fermentation broth 314, which is separated into ethanol 317 as a target biochemical and whole stillage 319. Whole stillage includes protein (e.g., plan3 protein (e.g., corn protein) and/or yeast protein from spent yeast cells), oil, fiber, residual carbohydrate (e.g., cellulose, starch, and the like), vitamins, minerals, and combinations thereof.

Whole stillage 319 can be separated into thin stillage and wet cake using one or more solid-liquid separators 320. Non-limiting examples of solid-liquid separators include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, filter device having one or more membrane filters), one or more screen devices (e.g., a “DSM” screen; one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof. As shown in FIG. 3, whole stillage is fed to solid-liquid separator 320 (e.g., decanter), or multiple decanters in parallel, to separate whole stillage 319 into wet cake 322 and thin stillage 325. The wet cake 322 is dried in dryer system 360 to form dried distiller's grain with solubles (DDGS) 361.

A portion 326 of the thin stillage 325 is transferred to the slurry tank system 250 as backset, while the rest 327 of the thin stillage 325 is transferred to an evaporation train that may include 4 to 8 evaporators in series (depending on plant size) to remove water and form syrup.

As shown in FIG. 3, the evaporation train includes four evaporators 331, 334, 337, and 340. The rest 327 of the thin stillage 325 is first fed to evaporator 331 to separate moisture as water vapor stream 332 and form a concentrated thin stillage 333. The concentrated thin stillage 333 is fed to evaporator 334 to separate moisture as water vapor stream 335 and form a concentrated thin stillage 336. The concentrated thin stillage 336 is fed to evaporator 337 to separate moisture as water vapor 338 and form a semi-concentrated syrup 339. Prior to reaching the end of the evaporator train the semi-concentrated syrup 339 (“skim feed”) is sent to a corn oil separation system, which removes corn oil product 392.

First, the semi-concentrated syrup 339 is separated in a “skim” centrifuge 373 into an emulsion 378 and defatted syrup 374. The defatted syrup 374 is sent to the final evaporator 340 in the evaporator train to separate moisture as water vapor stream 60 341 to form syrup 359, which is sent to dryer system 360 along with wet cake 322 to form DDGS 361. The emulsion 378 is combined with caustic 382 in emulsion tank 380 to help “break” the emulsion into an oil phase and aqueous phase that are more easily separated from each other. The treated emulsion 384 is pumped to oil centrifuge 386, wherein the treated emulsion 384 is separated into a corn oil product 392 and defatted emulsion (DFE) 388. The defatted emulsion 388 can accumulate in defatted emulsion (DFE) tank 389 and defatted emulsion 391 can be pumped via pump 390 to any desired location. As shown in FIG. 3, the defatted emulsion 391 is sent to dryer system 360 along with wet cake 322 and syrup 359 to form DDGS 361. Also, all of the water vapor streams 332, 335, 338 and 341 are combined into a single distillate stream 342.

The skim centrifuge 373 and oil centrifuge 386 can be centrifuges such as disk-stack centrifuges. In some embodiments, the skim centrifuge 373 and/or the oil centrifuge 386 can be configured to continuously or intermittently discharge accumulated solid particles (referred to as “discharges”). As shown in FIG. 3, discharges 387 from oil centrifuge 386 are combined with defatted emulsion 388 in DFE tank 389.

### EXAMPLES

### Example 1

Example 1 evaluated the removal of DON from different samples of corn using room-temperature reverse osmosis (RO) water.

A sample of DON-contaminated, whole corn was obtained from an ethanol plant. The sample of whole corn was split into three samples: one was left as whole kernels, one was coarsely milled using a laboratory disc mill, and the last was finely milled through a 0.5 mm screen with a Perten 3100 hammer mill. 100 g of each sample was mixed with 200 ml (about 200 grams) of room-temperature RO water for about 60 seconds, and then vacuum filtered through a coarse filter paper (VWR 28313-080). The wash water to corn sample weight ratio was about 2:1.

The filtrate percent total solids (% TS) was determined, and the grain particle material (corn flour) that was retained on the filter was dried overnight at 40° C., ground, and submitted for DON analysis using a Neogen Raptor integrated analysis platform. DON analysis showed the following:
Unwashed whole corn had 27.4 ppm of DON;
Washed whole corn had 17.6 ppm of DON;
Washed coarsely milled corn had 14.8 ppm of DON; and
Washed finely milled corn had 4.6 ppm of DON.

### Example 2

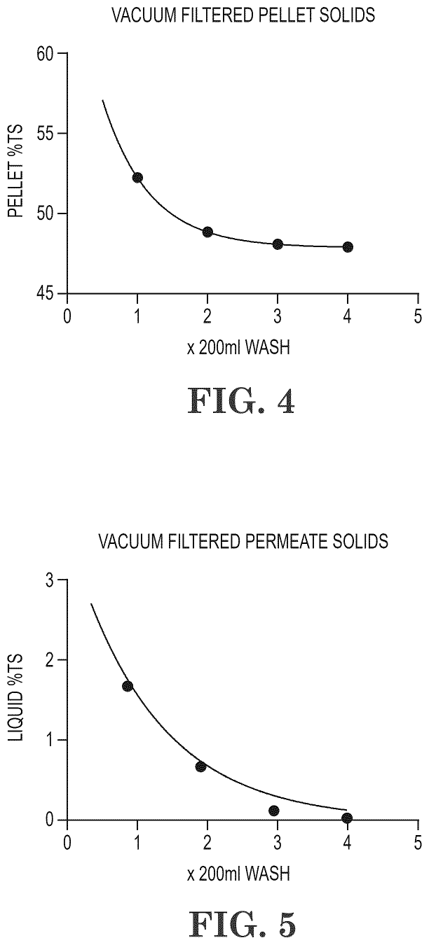

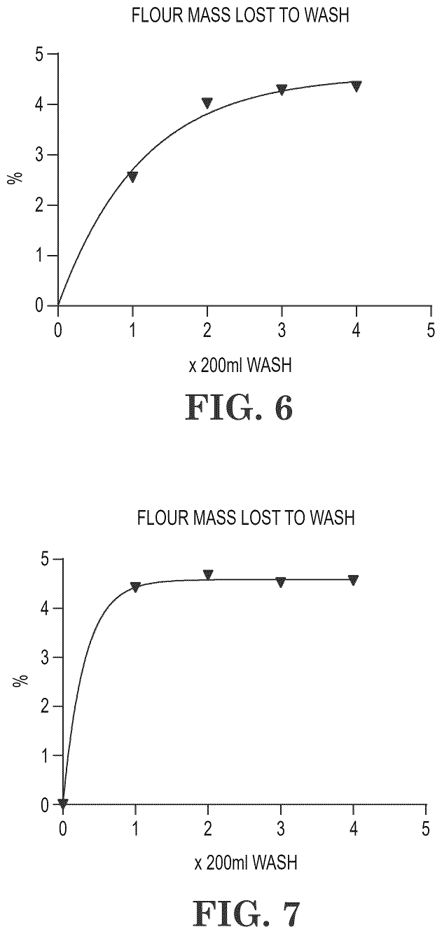

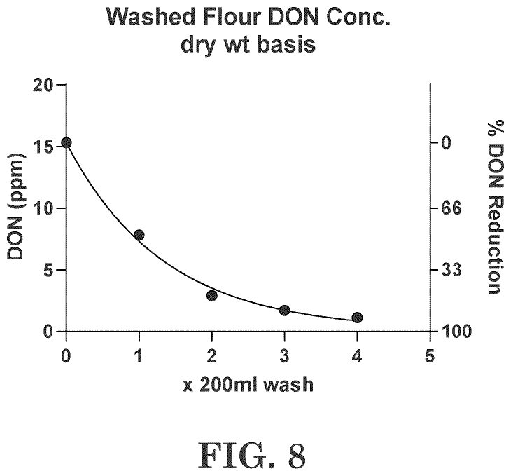

Example 2 evaluated a sample of corn flour after 1 to 4 wash cycles with room-temperature reverse osmosis (RO) water. A sample of DON-contaminated, whole corn was obtained from the same ethanol plant as in Example 1, and finely milled through a 0.5 mm screen with a Perten 3100 hammer mill to make corn flour. The sample of corn flour (grain particle material) was washed using a similar method as in Example 1. The water to corn sample weight ratio was about 2:1 for each wash cycle. 100 g of corn flour (as is) was mixed with 200 ml (about 200 g) of room temp RO water and filtered after mixing for about 60 seconds. The grain particle material (corn flour) that was retained on the filter was then resuspended with an additional 200 ml of RO water and filtered again. Samples of grain particle material (corn flour) that was retained on the filter after 1, 2, 3, and 4 wash cycles was dried at 40° C. and submitted for DON analysis (left side of FIG. 8), protein (FIG. 7), and percent total solids (% TS) (FIG. 4). The filtrate(liquid fraction) % TS (FIG. 5) was determined. The amount of material solubilized with each wash step was determined (FIG. 6). The amount of DON removed from each wash step was determined (right side of FIG. 8). As can be seen on the left side of FIG. 8, a wash water to corn flour sample weight ratio of 8:1 resulted in about a 93% reduction in DON (15.3 ppm to 1.1 ppm).

### Example 3

Example 3 evaluated different separation equipment that can be used after washing corn flour. A sample of DON-contaminated, whole corn was obtained from the same ethanol plant as in Example 1, and finely milled through a 0.5 mm screen with a Perten 3100 hammer mill to make corn flour.

A sample of corn flour (grain particle material) was washed using a similar method as in Example 1. The water to corn sample weight ratio was about 8:1. Using a Buchner funnel and vacuum filtration had resulted in a ˜50% solids cake.

A laboratory decanting centrifuge obtained from Lemitec GmbH was also evaluated. A wash slurry (aqueous composition) was made using 2 kg of corn flour and 16 kg of room temperature RO water, which corresponds to a water to corn flour weight ratio of 8:1. The wash slurry was mixed for 1 to 3 minutes prior to decanting. The decanter was set to 4400 xg bowl speed, with a scroll offset speed of 200 rpm, and using a 10 mm liquid retention ring. The centrifuge was fed at about 2800 ml/min of feed. The decanter was initially filled with water and was switched to the wash slurry (aqueous composition) to not overload with dry starchy material during startup. The resulting solids fraction (grain particle material after separating) was 52.28 % total solids and the resulting centrate (liquid fraction) was 0.46% total solids. It is noted that when slowing the scroll, the centrifuge torque quickly climbed and resulted in the centrifuge overloading. Example 3 demonstrated that the decanter resulted in cake solids comparable to filtration. The cake contains no free liquid and is likely the driest achievable without some sort of mechanical squeezing such as with a screw press. Decanting or filtration are approximately equivalent in terms of cake solids.

### Example 4

Example 4 evaluated different water to corn flour weight ratios.

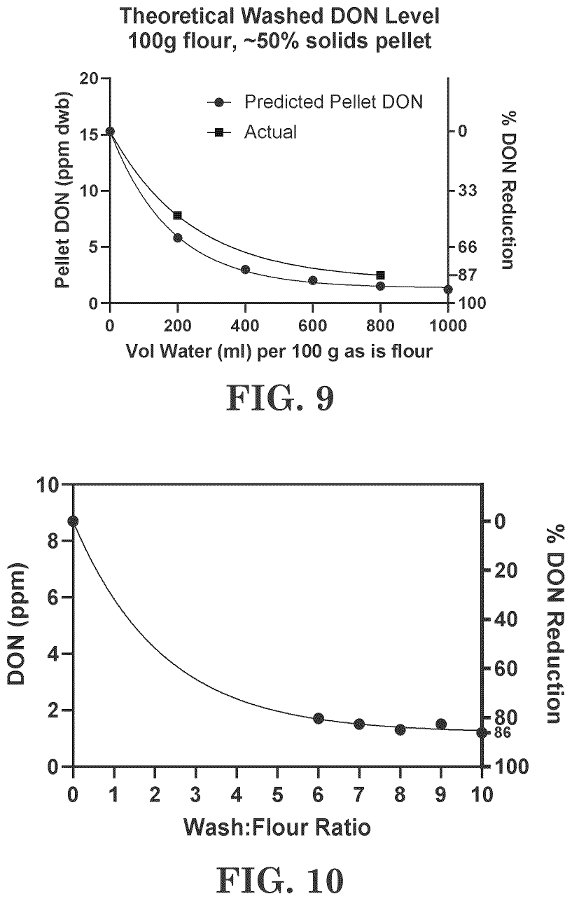

A single larger wash volume may be used at a biorefinery, so Example 4 was conducted to estimate the volume of a single wash that would achieve similar DON reductions as compared to multiple, sequential washes. FIG. 9 shows theoretical DON concentrations of different water to corn flour weight ratios assuming a 50% solids cake. Actual data was generated for samples having water to corn flour weight ratios of 200 ml(200 g):100 g and 800 ml(800 g):100g using the same process in Example 3 with respect to the laboratory decanting centrifuge obtained from Lemitec GmbH. The wash slurry was mixed for one minute prior to decanting. As can be seen in FIG. 9, the resulting actual DON reduction was higher than predicted, but follows the general trend.

Additional samples having water to corn flour weight ratios of 6:1, 7:1, 8:1, 9:1 and 10:1 were separated like the two actual samples in FIG. 9. The wash slurry was mixed for one minute prior to decanting. The results are shown in FIG. 10. As can be seen, a single large wash is approximately as effective as multiple smaller washes. Both methods achieve 85-90% reduction in DON using about 800 ml of wash liquid.

### Example 5

Example 5 evaluated the ability of ozone to treat the liquid fraction (water) after washing to reduce the concentration of DON after washing corn flour with a high level of DON. Ozone was generated using a TS-20 ozone generating system from Ozone Solutions producing about 5 l/min of a 5.5% ozone stream. A liquid fraction sample was prepared by forming a mixture of water and corn flour in a water to corn flour weight ratio of 2:1. The mixture was stirred for 60 seconds and then filtered using a Buchner funnel and vacuum filtration. After separating the liquid fraction from the mixture, the liquid fraction was treated with ozone for 20 minutes by bubbling the ozone gas stream through a flask that contained the liquid fraction. Samples of the liquid fraction before and after treatment with ozone were submitted to Trilogy Analytical Laboratories LC-MS for DON analysis. This ozone treatment reduced the DON in the liquid fraction from 6.7 ppm in the liquid fraction prior to ozone treatment to 1.4 ppm in the ozone-treated liquid fraction, which corresponds to about an 80% reduction in DON.

A second sample of liquid fraction that was more dilute in DON was evaluated for a longer exposure time to the ozone gas stream. The second sample was prepared by forming a mixture of water and corn flour in a water to corn flour weight ratio of 8:1. The mixture was stirred for 60 seconds and then filtered using a Buchner funnel and vacuum filtration. After separating liquid fraction from the mixture, the liquid fraction was treated with ozone for 2 hours by bubbling the ozone gas stream through a flask that contained the liquid fraction. The liquid fraction before and after treatment with ozone were submitted to Trilogy Analytical Laboratories LC-MS for DON analysis. This ozone treatment reduced the DON in the liquid fraction from 2.5 ppm in the liquid fraction prior to ozone treatment to below the lower reporting level (<0.1 ppm) in the ozone-treated liquid fraction.

This example demonstrated that ozone breaks down DON to low levels.

### Example 6

Example 6 evaluated using corn flour (grain particle material) that was washed by a washing process according to the present disclosure in a fermentation process that converts starch to ethanol. 250 ml bottle-scale fermentations were performed in 500 ml media storage bottles, with a 1/16^{th} inch hole drilled in the lid for venting. Fermentations were performed with a slurry that included corn flour. One sample of corn flour was made by grinding whole corn having high levels of DON in a 0.5 mm hammer mill. Another sample of corn flour was made by grinding the same whole corn having high levels of DON in a 0.5 mm hammer mill followed by washing the corn flour. The corn flour was washed by combining room temperature RO water and corn flour in a weight ratio of 8:1 to form a mixture. The mixture was stirred for 60 seconds and corn flour that had been washed was separated from the mixture in a decanting centrifuge like in Example 3.

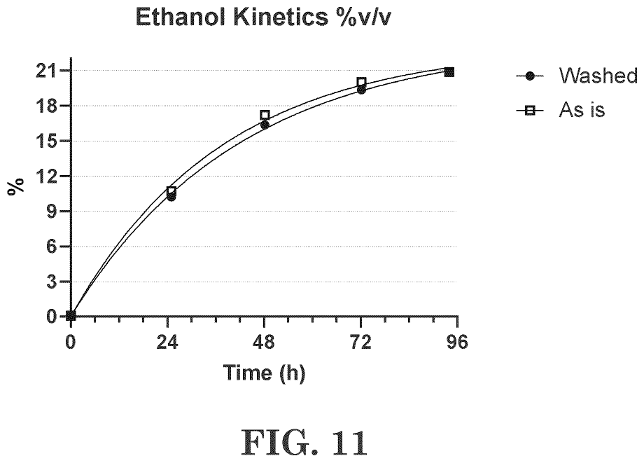

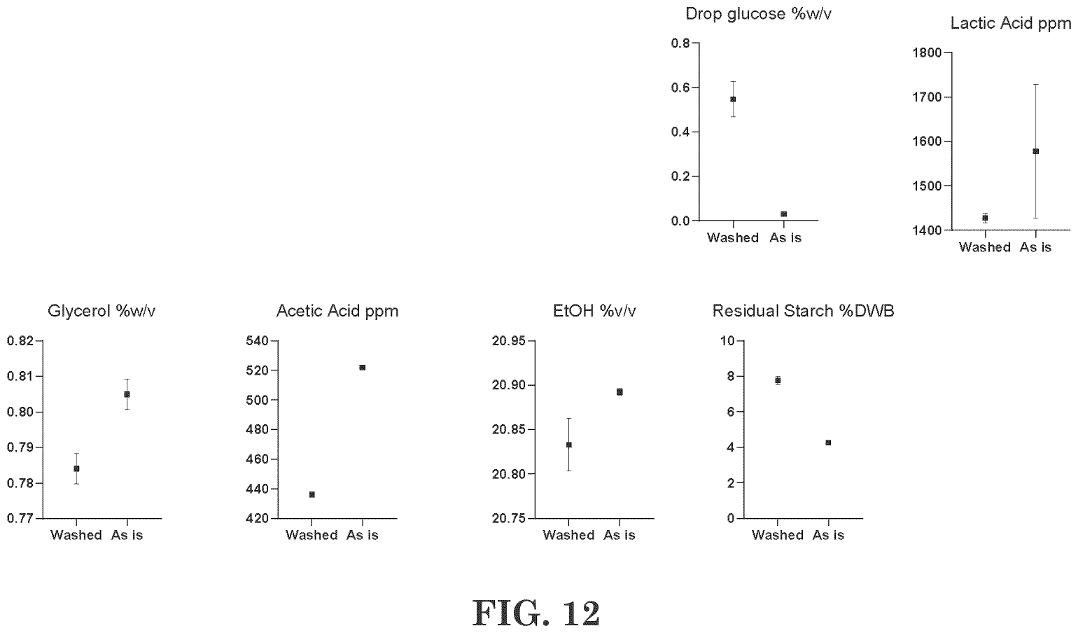

The fermentations were loaded to the equivalent total solids, and all other additions (backset, antimicrobial, enzyme, yeast, etc.) were equivalent. The fermentations were sampled at approximately 24-hour intervals for HPLC analysis of sugars, and ethanol. The final drop samples were also analyzed for residual starch. The results are shown in FIGS. 11 and 12. Fermentation followed similar ethanol kinetics and achieved similar final ethanol titer. Fermentation performance is roughly equivalent. Reduced lactic acid acetic acid concentrations from the washed flour suggests that there is some antimicrobial effect.

### Example 7

Example 7 evaluated using corn flour (grain particle material) that was washed by a washing process according to the present disclosure in a fermentation process that converts starch to ethanol. 250 ml bottle-scale fermentations were performed in 500 ml media storage bottles, with a 1/16^{th} inch hole drilled in the lid for venting. Fermentations were performed with a slurry that included corn flour. One sample of corn flour was made by grinding whole corn having high levels of DON in a 0.5 mm hammer mill. Another sample of corn flour was made by grinding the same whole corn having high levels of DON in a 0.5 mm hammer mill followed by washing the corn flour. The corn flour was washed by combining room temperature biorefinery process water streams and corn flour in a weight ratio of 8:1 to form a mixture. The mixture was stirred for 60 seconds, and corn flour that had been washed was separated from the mixture in a floor model centrifuge.

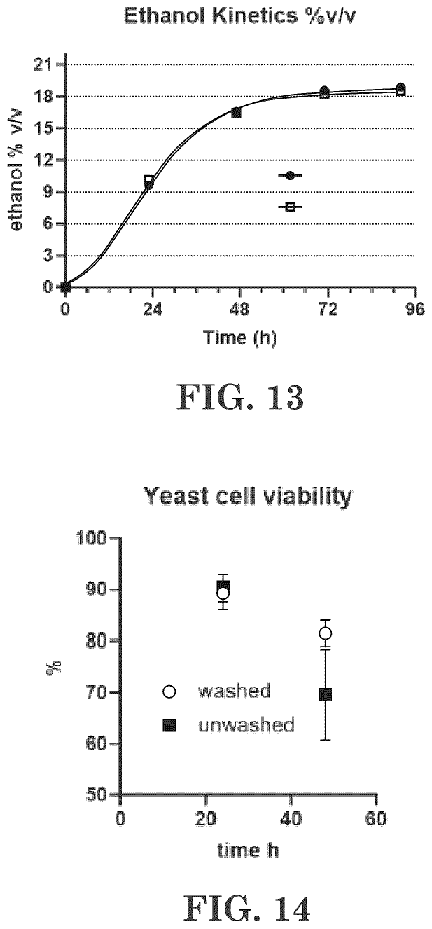

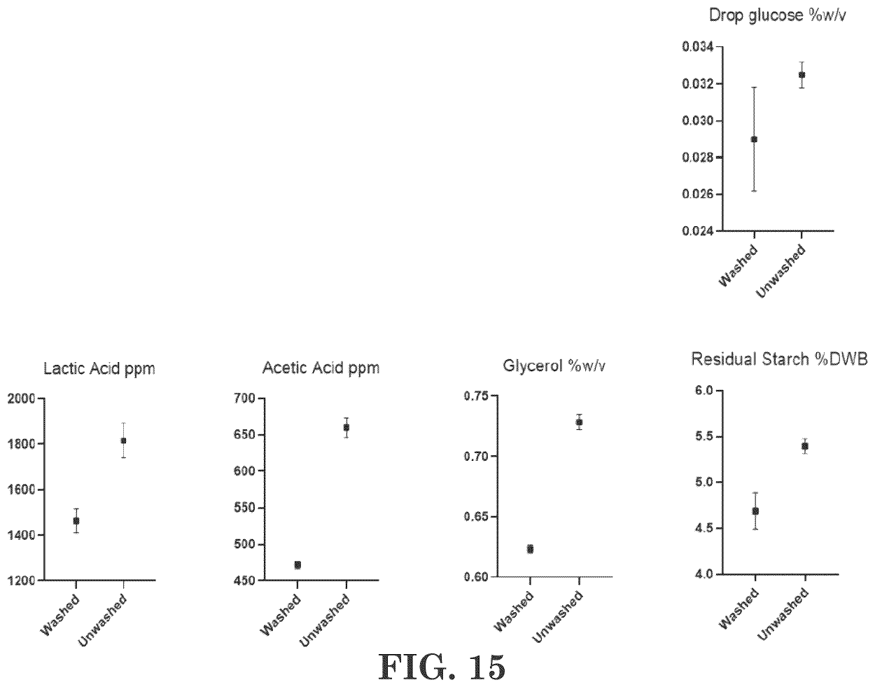

The fermentations were loaded to the equivalent total solids, and all other additions (backset, antimicrobial, enzyme, yeast, etc.) were equivalent. The fermentations were sampled at approximately 24-hour intervals for HPLC analysis of sugars, and ethanol. The final drop samples were also analyzed for residual starch. The results are shown in FIGS. 13 to 15.

Reduced glycerol, improved sugar consumption based on the final drop samples, higher yeast cell viability may all be indicators of improved yeast health in this process. While not being bound by theory, these results may be due to reduced organic acid stress or other factors related to ozonation or hydration of flour solids during washing before the slurring process.

### Example 8

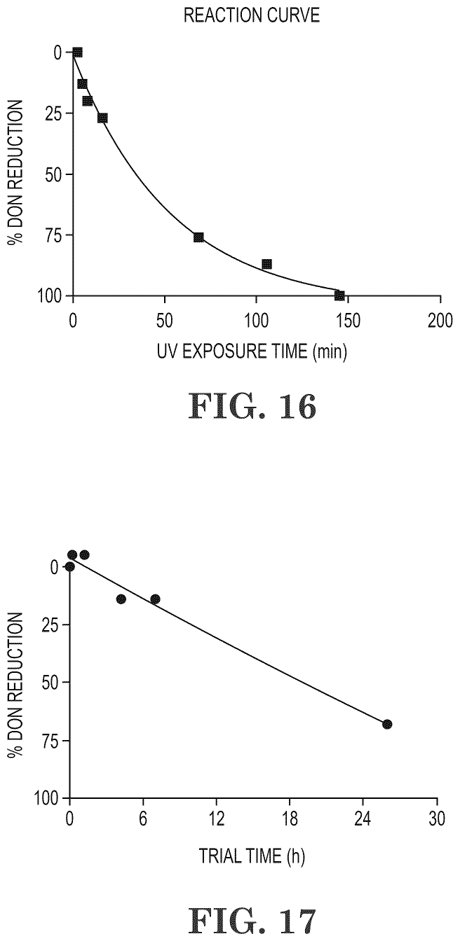

Example 8 demonstrated at a lab-bench scale that ultraviolet light can reduce DON in wash water after washing corn flour and separating the wash water from solids. A sample of DON contaminated corn flour was obtained from a dry-grind corn ethanol pilot facility. 100 g of the corn flour was mixed with 800 ml of reverse osmosis (RO) water (weight ratio of the RO water to the corn flour was about 8:1) for about 60 seconds, and then vacuum filtered through a 20 μm filter paper using Buchner funnel to produce a filtrate. 600 ml of the filtrate was recycled through a UV sterilization system for 4 hours at 600 ml/min flow rate at room temp. The internal UV chamber volume was 300 ml. The UV lamp output was 11 watts (W). Samples were taken at set time points and submitted for DON analysis. The exposure time was determined based on the flow rates and volumes used, and the results are shown in FIG. 16.

The UV sterilization system was an iSpring UVF11A inline UV system having an 11 Watt UV lamp.

### Example 9

Example 9 demonstrated at a pilot-facility scale that ultraviolet light can reduce DON in wash water after washing corn flour and separating the wash water from solids. The trial objective was to investigate the UV exposure time in terms of trial time and recirculation condition that resulted in reduced DON concentrations within the filtrate. A relatively large sample of high DON filtrate was generated by mixing a slurry of 53.8 lbs of high DON corn flour and 50 gallons of water (weight ratio of the water to the corn flour was about 7.8:1), and then filtering the slurry through a 2 micron filter press to generate the filtrate. For the UV treatment, 5 gallons of the filtrate was used to determine the efficacy of the UV lamp treatment time using a SQ8-PA 37 UV treatment system having a 37 watt UV lamp. The filtrate was recirculated between the UV lamp chamber and a residence tank using a peristaltic pump at a flow rate of 0.3 GPM to ensure adequate UV exposure. Samples were taken at different set points throughout the 26-hour trial and submitted for DON analysis. The results are shown in FIG. 17.

### Example 10

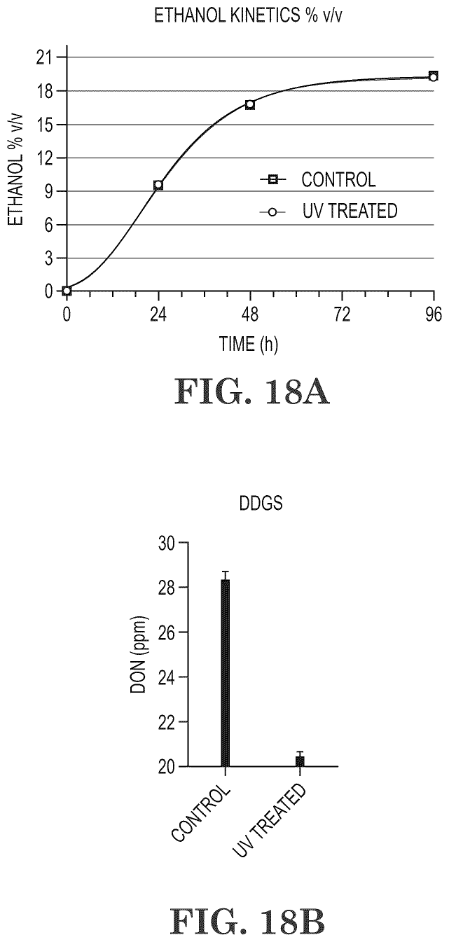

Example 10 evaluated fermentations to determine if the washing process in Example 8 has an impact on the fermentation process. 250 ml bottle scale fermentations were performed in 500 ml media storage bottles, with a 1/16^{th} inch hole drilled in the lid for venting. Fermentation bottles were loaded with high DON flour that had been washed and filtered with 8:1 water to flour weight ratio as described in Example 8. The fermentation bottles were loaded to the equivalent total solids, and all other additions (backset, antimicrobial, enzyme, yeast, etc.) were equivalent. The backset contained thin stillage and wash water (either untreated for the control or UV treated for the test) from the flour washing process in Example 8. In the control fermentation the wash water that was not treated with UV was recombined with washed flour. In test fermentation the wash water was exposed to ultraviolet in the UV treatment system for 120 minutes of exposure time using the UV treatment system in Example 8. UV-treated wash water was recombined with washed flour to create slurry. The fermentations were sampled at approximately 24-hour intervals for HPLC sugars, and ethanol. The final drop samples were also analyzed for residual starch. The results are shown in FIGS. 18A and 18B. No significant differences were noted between the fermentations in FIG. 18A. Ethanol kinetics and residuals at the end of the fermentation were comparable. DDGS produced from the treated filtrate contained less DON in the final product as shown in FIG. 18B.

Being able to wash corn flour at the front end advantageously reduces the DON level in DDGS while still being able to produce a DDGS product in a manner that can satisfy product specifications.

### Example 11

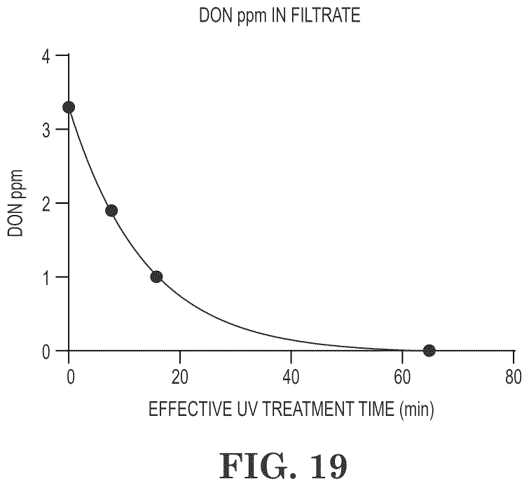

Example 11 evaluated UV treatment of DON wash water filtrate with the addition of hydrogen peroxide as an oxidation catalyst. A sample of DON contaminated corn flour was obtained from a dry-grind corn ethanol pilot facility. 100 g of the corn flour was mixed with 800 ml of RO water for about 60 seconds, then vacuum filtered through a 20μm filter paper using Buchner funnel. 6 ml of 37% hydrogen peroxide was added to 600 ml of generated filtrate. The filtrate was immediately recycled through a UV sterilization system model PL160520 for 300 minutes at 600 ml/min flow rate at room temp. The internal UV chamber volume was 300 ml. The UV lamp output was 11 W. Samples were taken at set time points and submitted for DON analysis. The exposure time (“effective UV treatment time) was determined based on the flow rates and volumes used. The results are shown in FIG. 19. Addition of peroxide achieved 100% DON reduction in about 65 minutes of effective UV exposure. 100% DON reduction in the same system, but without peroxide addition, achieved 100% DON reduction in about 150 minutes of UV exposure as shown in FIG. 16. Addition of hydrogen peroxide at 20% v/v to filtrate, but without UV treatment, resulted in only 13% reduction in DON after 300 minutes of room temperature incubation. This indicates the addition of hydrogen peroxide, a strong oxidizer, improves overall DON treatment in a UV system. Ultraviolet light breaks down hydrogen peroxide molecules creating hydroxyl radicals - strong oxidizers, breaking down DON molecule bonds.

1. A method of washing a grain particle material, wherein the method comprises:
providing an aqueous composition comprising:
the grain particle material; and
an aqueous liquid;
separating the aqueous composition into a solids fraction and at least one liquid fraction; and
combining the solids fraction with water to form a slurry comprising grain starch.

2. The method of claim 1, wherein the grain particle material comprises one or more mycotoxins, and wherein the grain particle material comprises grain flour.

3. (canceled)

4. The method of claim 2, wherein the grain flour is formed by exposing a whole grain to one or more particle size reduction processes, and wherein the whole grain is chosen from corn, wheat, barley, oat, sorghum, rice, millet, and combinations thereof.

5. (canceled)

6. The method of claim 1 further comprising:
hydrolyzing at least a portion of grain starch in the slurry into one or more monosaccharides to form a fermentable composition;
fermenting the fermentable composition to form a fermented composition comprising one or more biochemicals, wherein the one or more biochemicals comprise ethanol, and further comprising separating ethanol from the fermented composition;
separating ethanol from the fermented composition; and
forming an animal feed product from at least a portion of the fermented composition.

7. (canceled)

8. (canceled)

9. The method of claim 1, wherein the aqueous liquid is at a temperature below a starch-gelatinization temperature of the grain starch.

10. The method of claim 1, wherein the aqueous liquid is at a temperature of 55°C. or less.

11. The method of claim 1, wherein the solids fraction has a total solids content of 40 percent or more by total weight of the solids fraction.

12. The method of claim 1, wherein providing an aqueous composition comprises:
providing the aqueous liquid;
providing the grain particle material, wherein the grain particle material comprises the one or more mycotoxins in a first concentration; and
combining the aqueous liquid and the grain particle material in a weight ratio of 5:1 or more,
wherein the solids fraction has a second concentration of the one or more mycotoxins that is less than the first concentration.

13. The method of claim 12, wherein the second concentration is 25 percent or less of the first concentration.

14. The method of claim 1, further comprising, prior to separating, mixing the aqueous composition for a time period of at least 30 seconds.

15. The method of claim 1, wherein the at least one liquid fraction has a concentration of the one or more mycotoxins, and further comprising treating the at least one liquid fraction to reduce the concentration of the one or more mycotoxins in the at least one liquid fraction.

16. The method of claim 15, wherein treating the at least one liquid fraction comprises exposing the at least one liquid fraction to one or more oxidants that modify a molecular structure of the one or more mycotoxins, and wherein the one or more oxidants are chosen from hydroxyl radicals, hydrogen peroxide, ozone, superoxide anions, chlorine dioxide, persulfates, and combinations thereof.

17. (canceled)

18. The method of claim 15, wherein treating the at least one liquid fraction comprises:
filtering the at least one liquid fraction into a retentate and permeate, wherein the retentate is more concentrated in the one or more mycotoxins as compared to the at least one liquid fraction; and
exposing the retentate to one or more oxidants that modify a molecular structure of the one or more mycotoxins.

19. The method of claim 15, wherein treating the at least one liquid fraction comprises adding an enzyme composition to the at least one liquid fraction, wherein the enzyme composition comprises at least one enzyme having an activity to detoxify at least one mycotoxin.

20. The method of claim 15, wherein treating the at least one liquid fraction comprises exposing the at least one liquid fraction to ultraviolet radiation to modify a molecular structure of the one or more mycotoxins.

21. The method of claim 15, further comprising, after treating, recycling at least a portion of the at least one liquid fraction to form the aqueous composition.

22. The method of claim 15, further comprising:
after treating, recycling at least a portion of the at least one liquid fraction to form the aqueous composition;
hydrolyzing at least a portion of the grain starch into one or more monosaccharides to form a fermentable composition; and
fermenting the fermentable composition to form a fermented composition comprising one or more biochemicals.

23. The method of claim 22, further comprising forming an animal feed product from the fermented composition.

24. The method of claim 1, wherein the aqueous composition is separated into the solids fraction and the at least one liquid fraction via one or more solid-liquid separators chosen from one or more centrifuges one or more decanters, one or more filters one or more screen devices, one or more brush strainers, one or more vibratory separators, one or more hydrocyclones, and combinations thereof.

25. The method of claim 1, wherein the aqueous liquid is chosen from water, one or more process streams in a biorefinery, and combinations thereof.

26. The method of claim 2, wherein the one or more mycotoxins are chosen from one or more aflatoxins, one or more ochratoxins, patulin, one or more fumonisins, one or more zearalenones, one or more trichothecenes, and combinations thereof.

27. The method of claim 2, further comprising determining if the one or more mycotoxins are present in incoming grain and/or one or more biorefinery process compositions.

28. The method of claim 2, wherein the one or more mycotoxins comprise one or more water-soluble mycotoxins.

29-57. (canceled)

58. A method comprising:
grinding corn to produce corn flour;
combining the corn flour with water to form a first slurry;
separating the first slurry into a solids fraction and at least one liquid fraction;
treating the at least one liquid fraction to reduce a concentration of one or more mycotoxins in the at least one liquid fraction;
after the treating, recycling at least a portion of the at least one liquid fraction to form the first slurry;
combining the solids fraction with water to form a second slurry;
hydrolyzing at least a portion of corn starch in the second slurry into one or more monosaccharides to form a fermentable composition;
fermenting the fermentable composition to form a fermented composition comprising ethanol; and
forming an animal feed product from at least a portion of the fermented composition.

59. The method of claim 58, wherein the hydrolyzing and fermenting can occur simultaneously in a fermentation reactor, or sequentially in one or multiple fermentation reactors.
